(19) United States
(12) Patent Application Publication
(10) Pub. No.: US 2024/0020529 A1
(43) Pub. Date: Jan. 18, 2024

(54) PCT/US21/056841

(71) Applicant: ISP INVESTMENTS LLC, Wilmington, DE (US)

(72) Inventors: Yunxia BI, Garnet Valley, PA (US); Thomas DURIG, West Chester, PA (US); Solomon Howard JACOBSON, Berkeley Heights, NJ (US)

(73) Assignee: ISP INVESTMENTS LLC, Wilmington, DE (US)

(21) Appl. No.: 18/034,149

(22) PCT Filed: Oct. 27, 2021
(86) PCT No.: PCT/US21/56841

§ 371 Date: Apr. 27, 2023

Related U.S. Application Data
(60) Provisional application No. 63/106,212, filed on Oct. 27, 2020.

(51) Int. Cl. G06N 3/08

(52) U.S. Cl. CPC G06N 3/08

(57) ABSTRACT

Present invention is directed to an artificial intelligence-based method and a system, that uses both experimental results data and molecular simulation to predict properties of amorphous solid dispersions such as glass transition temperature, dissolution profile, and/or physical stability. The method and system of the present invention enable rational design of amorphous solid dispersions for poorly water-soluble drugs and significantly reduce the time and resource required for amorphous solid dispersions formulation development.

### CROSS-REFERENCE TO RELATED APPLICATIONS

This International Patent Application claims priority to U.S. Provisional Application No. 63/106,212 filed on Oct. 27, 2020, which is incorporated herein by reference in their entireties.

### FIELD OF THE INVENTION

The present application relates to a method for predicting properties of amorphous solid dispersions using artificial intelligence and machine learning techniques, and more particularly to a method and system for predicting properties of amorphous solid dispersions such as glass transition temperature, dissolution profile, and/or physical stability, using both experimental results data and molecular simulation.

### BACKGROUND OF THE INVENTION

Amorphous active pharmaceutical ingredients demonstrate higher apparent water solubilities, and thus can effectively improve the bioavailability of poorly water soluble active pharmaceutical ingredients (APIs). Meanwhile, due to their metastable nature, amorphous APIs are prone to crystallization during storage and upon dissolution in gastrointestinal tracts. A large proportion of newly discovered APIs display poor solubility in the gastrointestinal fluids, which tends to decrease their bioavailability. To improve the aqueous solubility of APIs, different formulation methods have been designed including amorphous forms, which have no long-range crystallographic order and higher internal energy compared with their respective crystalline forms. However, pure amorphous APIs are often physically unstable and can crystallize as a result of increased molecular mobility, especially when stored above their glass transition temperature or in humid environments. A popular method to improve the stability of amorphous APIs is to include the API in the form of an amorphous solid dispersions (ASDs).

Inert carriers are often used in stabilized amorphous APIs. A successful amorphous solid dispersion (ASD) can maintain physical stability in solid dosage form as well as exhibit fast dissolution and sustain supersaturation in gastrointestinal tract for an extended time period. Amorphous solid dispersions are formed by (molecularly) dispersing an API in a (usually amorphous) polymer, which acts as an inactive stabilizer. Stabilization (even above the solubility limit of the API in the polymer) is caused by the polymer increasing the glass transition temperature and forming intermolecular interactions, which in turn results in reduced molecular

However, proper selection of a polymeric carrier and/or other ingredients of an amorphous solid dispersion formulation is a complex process, considering various adverse parameters associated with the polymer, drug, or other ingredients, that directly or indirectly affect the physical stability and dissolution behaviors of resulting amorphous solid dispersions. Currently, the manual testing and evaluation to assess properties of amorphous solid dispersion such as physical stability and dissolution profiles with respect to given ingredient(s) involves multiple trial-and-error experiments and may take several months, typically about three to six months. It is extremely time-consuming and most of the times the results are unpredictable. If unsuccessful, the long cycle has to be repeated and re-tested with different experimental conditions or different combinations of drug, polymer and/or other ingredients. Moreover, theoretical models need large amount of physicochemical information of each component and substantial professional knowledge. The prediction capability of these models has been quite limited with the uncontrolled error due to the mathematic hypothesis.

Machine learning is a branch of artificial intelligence, that extensively finds applications in pharmaceutical research industry and formulation design. Machine learning algorithms implement tools like artificial neural networks, that can learn from a training data set and subsequently used to predict complex systems. Various approaches have been discussed in the prior-art that use the benefits of artificial intelligence and machine learning techniques in formulation development and prediction.

Run Han et. al. discloses a physical stability prediction system using machine learning techniques wherein this prediction model studies around eight machine learning approaches and identifies random forest (RF) model, that has achieved the best prediction accuracy for physical stability of the solid dispersion formulations.

Kok Khiang Peh et. al. discloses the use of artificial neural networks to predict drug dissolution profiles. However, this study teaches the prediction of dissolution profiles of matrix-controlled release theophylline pellet preparation and evaluates network performance by comparing the predicted dissolution profiles with those obtained from physical experiments using similarity factor.

Juliet Obianuju Njoku et. al. discloses a software based system for predicting excipient influence on dissolution profiles involving amorphous solid dispersion systems. The objective of this study is to assess formulation specific models in simulating drug-excipient interaction using DDDPlus, by determining the impact of prediction factors in the program on solubilizer and disintegrant effect on the dissolution profile of an immediate release, poorly soluble drug.

U.S. Pat. No. 10,216,911 B2 discloses a method for predicting compound solubility, involving calculating a free energy of solubility for a compound in a solvent, where an initial state is established for a system by a computer model that contains an aggregate of multiple molecules.

WO 2020/016,579 A2 discloses a machine-learning based, method of analyzing drug-like molecules, that involves representing molecular quantum states of each drug-like molecule as a quantum graph, and feeding that quantum graph as input to machine learning system

In view of foregoing disclosures, still there is a need to establish a reliable system for amorphous solid dispersion design, that helps to predict various properties of ASDs.

In the present invention, an artificial intelligence-based system is disclosed using both experimental results data and molecular simulation to predict various properties of ASDs. This system enables rational design of ASDs for poorly water-soluble drugs and may significantly reduce the time and resources required for ASD based formulation development.

### SUMMARY OF THE INVENTION

In some implementations, properties of amorphous solid dispersions are predicted by a method performed at an electronic device having a processor. The method obtains a machine learning model trained to predict a dissolution, thermophysical, or stability property of an amorphous solid dispersion based on at least one parameter of at least one first ingredient. The machine learning model was trained based on comparing a predicted dissolution, thermophysical, or stability property predicted based on the at least one first ingredient with an experimentally-determined dissolution, therrnophysical, or stability property that is experimentally determined using the at least one first ingredient. The method determines at least one parameter of at least one second ingredient of a second amorphous solid dispersion and predicts at least one dissolution, thermophysical, or stability property of the second amorphous solid dispersion by inputting the at least one parameter of the at least one second ingredient to the machine learning model.

In some implementations, a machine learning model is trained to predict properties of amorphous solid dispersions by performing steps comprising: (i) creating a plurality of experimental results data of at least one first ingredient of an amorphous solid dispersion; (ii) generating molecular simulation properties of at least one first ingredient of the amorphous solid dispersion of step (i); (iii) implementing a machine learning model (e.g., an artificial neural network) using experimental results data of step (i) and molecular simulation properties of step (ii). The machine learning model is used to predict the properties of amorphous solid dispersions comprising at least one second ingredient. The machine learning model may be updated over time with additional training, e.g., based on new experimental result and/or molecular simulation data.

In another aspect, the ingredient of the amorphous solid dispersion is selected from the group consisting of polymers, drugs, sugars, sugar alcohols, surfactants, organic acids and bases, inorganic molecules, co-solvents, co-excipients, plasticizers, and combinations thereof.

In another aspect, the ingredient of the amorphous solid dispersion is selected from at least one drug and at least one polymer.

In another aspect, the predicted properties of the amorphous solid dispersions include glass transition temperature, physical stability, maximum drug concentration during dissolution in Fasted State Simulating Intestinal Fluid [FaSSIF (Cₘₐₓ)], and drug concentration at 120 min during dissolution in Fasted State Simulating Intestinal Fluid [FaSSIF (C₁₂₀)].

Yet another aspect of the present application is to provide a system for predicting properties of the amorphous solid dispersions comprising at least one computer system capable of executing the steps of: (i) receiving a plurality of experimental results data of at least one first ingredient of an amorphous solid dispersion; (ii) generating a plurality of two-dimensional or three-dimensional structures of at least one first ingredient of the amorphous solid dispersion of step (i); (iii) performing molecular simulation to generate molecular simulation properties of at least one first ingredient of the amorphous solid dispersion of step (ii); (iv) implementing an artificial neural network using experimental results data of step (i) and molecular simulation properties of step and (v) predicting the properties of amorphous solid dispersions comprising at least one second ingredient, using the artificial neural network of step (iv).

In another aspect, the computer system for predicting properties of the amorphous solid dispersions comprises (i) a memory configured to store at least one program, (ii) a processor (iii) a visualization interface, or combinations thereof.

In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein. In accordance with some implementations, a non-transitory computer readable storage medium has stored therein instructions, which, when executed by one or more processors of a device, cause the device to perform or cause performance of any of the methods described herein. In accordance with some implementations, a device includes: one or more processors, a non-transitory memory, and means for performing or causing performance of any of the methods described herein.

### BRIEF DESCRIPTION OF THE FIGURES

Further embodiments of the present application can be understood with reference to the appended figures.

FIG. 1 is a flow chart illustrating an exemplary process of predicting properties of amorphous solid dispersions using a trained artificial neural network.

FIG. 2a illustrates a block diagram describing the process of implementing an artificial neural network.

FIG. 2b illustrates a block diagram describing the process of predicting properties of amorphous solid dispersions using a trained artificial neural network.

FIG. 3 illustrates a graph representing actual versus predicted values of physical stability at 25° C./60% relative humidity.

FIG. 4 illustrates a graph representing actual versus predicted values of physical stability at 40° C./75% relative humidity.

FIG. 5 illustrates a graph representing actual versus predicted values of maximum drug concentration during dissolution in Fasted State Simulating Intestinal Fluid [FaSSIF (Cₘₐₓ)].

FIG. 6 illustrates a graph representing actual versus predicted values of drug concentration at 120 min during dissolution in FaSSIF/Maximum drug concentration during dissolution in FaSSIF (C₁₂₀/Cₘₐₓ).

FIG. 7 is a block diagram of an example system architecture of an exemplary device in accordance with some implementations.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such, as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions, and ratios as used herein, are by weight of the total composition, unless otherwise specified. All such weights as they pertain to listed ingredients are based on the active level and, therefore: do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

The term “polymer” refers to a compound comprising repeating structural units (monomers) connected by covalent chemical bonds. Polymers may be further derivatized, crosslinked, grafted or end-capped. Non-limiting examples of polymers include homopolymers, copolymers, terpolymers, tetra-polymers, quaternary polymers, ampholytic polymers, water soluble polymers, water in-soluble polymers, ionizable polymers, non-ionizable polymers, oligomers, and homologues. The term “copolymer” farther refers to a polymer consisting essentially of two or more different types of monomers polymerized to obtain, the copolymer.

The term “amorphous solid dispersion” refers to a system including an amorphous active pharmaceutical ingredient stabilized by an excipient, commonly a polymer and other optional ingredients to enhance the physical stability and dissolution behavior.

The term “active pharmaceutical ingredient” or “drug” refers to a medicine or pharmaceutically active substance which has a physiological effect when ingested or otherwise introduced into the body.

The term “experimental results data” refers to the test results data of various selected ingredients of amorphous solid dispersions, generated using standard test procedures and experiments.

The term “molecular simulation” refers to computational techniques to mimic molecular behavior of the ingredients of an amorphous solid dispersion at atomic level and simulate two dimensional or three-dimensional structures, that help to analyze various structural, dynamic, and energetic information.

The term “artificial neural network” refers to a computational architecture having programmed instructions that is capable of learning from a training data set to make one or more predictions such as predictions of properties of new test objects.

The term “computer system” refers to an electronic device that includes a memory configured to store coded instructions, a processor to execute the instructions, an output interface, etc., capable of performing various claimed steps of the present invention.

In a non-limiting embodiment, the present application discloses a method and system for predicting properties of amorphous solid dispersions such as glass transition temperature, dissolution profile, and/or physical stability, using both experimental results data and molecular simulation.

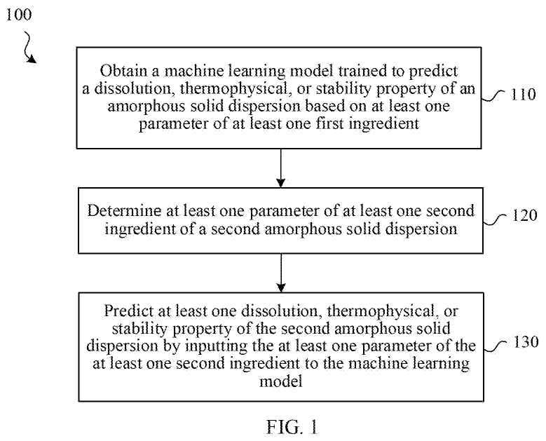

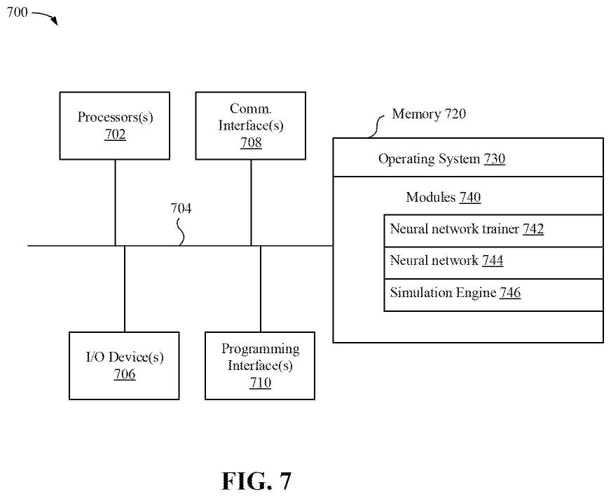

FIG. 1 is a flow chart illustrating an exemplary method 100 of predicting properties of amorphous solid dispersions using a trained artificial neural network. In some implementations, the method 100 is performed by a device (e.g., device 700 of FIG. 7). The method 100 can be performed at a mobile device, desktop, laptop, or server device. In some implementations, the method 100 is performed by processing logic, including hardware, firmware, software, or a combination thereof. In some implementations, the method 100 is performed by a processor executing code stored in a non-transitory computer-readable medium (e.g., a memory).

At block 110, the method 100 obtains a machine learning model trained to predict, a dissolution, thermophysical, or stability property of an amorphous solid dispersion based on at least one parameter of at least one first ingredient. The machine learning model was trained based on comparing a predicted dissolution, thermophysical, or stability property predicted based on the at least one first ingredient with an experimentally-determined dissolution, thermophysical, or stability property that is experimentally determined using the at least one first ingredient. For example, the machine learning model may be trained based inputting a property of two ingredients of an amorphous solid dispersion (e.g., a property of an API and a property of a polymer). During an iterative training process, the machine learning model may make predictions based on such input, compare those output predictions with experimentally-determined dissolution, thermophysical, or stability properties and adjust the configuration of the machine learning model to reduce differences between predictions and experimentally-known results in future iterations. For example, the weight values of neural network nodes of a neural network-type machine learning model may be adjusted to reduce errors and thus improve predictive accuracy. Training such a model using a number of amorphous solid dispersions (e.g., different API/polymer combinations) can provide a machine learning model that is accurate with respect to predicting dissolution/stability properties of many potential dispersions, including combinations of ingredients that were not necessarily used in the training process.

In some implementations, the at least one parameter of the at least one first ingredient includes a simulation, such as a simulation that provides molecular simulation. The at least one parameter may be determined utilizing molecular drawing tools such as ChemSketch available from Advanced Chemistry Development Inc. of Toronto Canada, ChemDraw available from PerkinElmer Inc. of Waltham, MA, PubChem Sketcher available at https://pubchem.ncbi.nlm.nih.gov//edit3/index.html, etc., molecular property prediction tools such as Molsoft® available from Molsoft L.L.C. or EPI Suite™, available from the U.S. Environmental Protection Agency, etc., and/or molecular simulation software such as Materials Studio available from Bovia of San Diego, California, Amber available at https://ambermd.org/AmberTools.php, LAMMPS available at https://www.lammps.org/, Gromacs available at http://www.gromacs.org/, etc.

Having obtained the trained machine learning model, the remaining elements of method 100 use the model to make a prediction for a second ingredient. At block 120, the method 100 determines at, least one parameter of at least one second ingredient of a second amorphous solid dispersion. At block 130, the method 100 predicts at least one dissolution, thermophysical, or stability property of the second amorphous solid dispersion by inputting the at least one parameter of the at least one second ingredient to the machine learning model. In some implementations, the at least one parameter of the at least one second ingredient, is an experimental API parameter such as a molecular weight, melting point, water solubility, or value associated with an experimental octanol water partition coefficient. In some implementations, the at least one parameter of the at least one second ingredient is a computed API parameter, such as a number of hydrogen bond acceptors and donors, a solubility value, or a molecular volume. In some implementations, the at least one parameter of the at least one second ingredient is a computed polymer parameter comprising a thermophysical property like glass transition temperature, density, surface tension, solubility parameters, etc., a mechanical property like modulus, Poisson's ratio, etc., or a geometrical property like monomer length, and volume, etc. In some implementations, the at least one parameter of the at least one second ingredient is an enthalpy of mixing, an API-polymer interaction energy, a polymer-water partition coefficient, or a solvation free energy of the drug molecule in the polymer.

In some implementations, a machine learning model such as an artificial neural network is trained to predict properties of amorphous solid dispersions by performing steps including: (i) creating a plurality of experimental results data of at least one first ingredient of an amorphous solid dispersion; (ii) generating molecular simulation properties of at least one first ingredient of the amorphous solid dispersion of step (i); (iii) implementing a machine learning model (e.g., an artificial neural network) using experimental results data of step (i) and molecular simulation properties of step (ii). The machine learning model is used to predict the properties of amorphous solid dispersions comprising at least one second ingredient. The machine learning model may be updated over time with additional training.

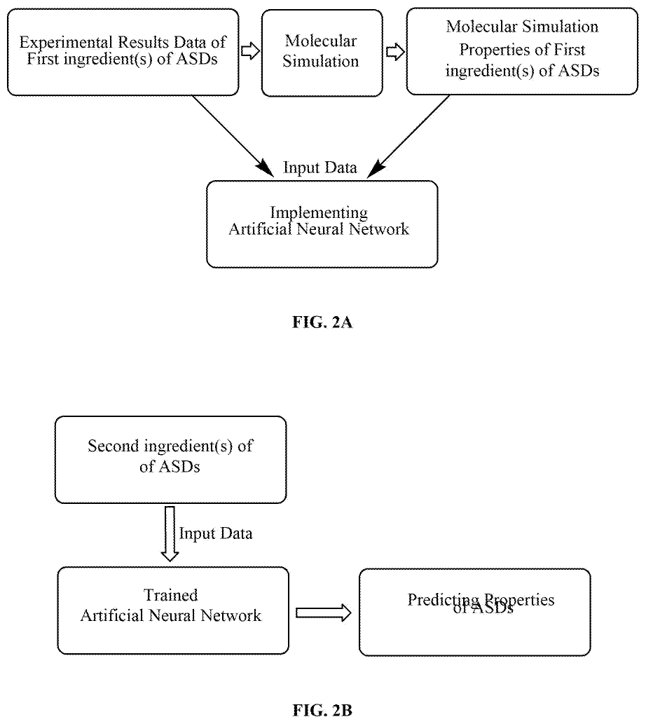

FIG. 2a illustrates a block diagram describing the process of implementing an artificial neural network. In this example, experimental result data of first ingredient(s) of ASDs are used to produce molecular simulations that simulate the properties of the first ingredients. The experimental results data and the molecular simulations are used in implementing the artificial neural network.

FIG. 2b illustrates a block diagram describing the process of predicting properties of amorphous solid dispersions using a trained artificial neural network. In this example, the second ingredient(s) of ASDs are input to the trained artificial neural network, which uses the input to predict properties of ASDs.

The second ingredient is different from the first ingredient of the amorphous solid dispersion, used for creating the experimental results data. Ingredients of amorphous solid dispersion are selected from the group comprising polymers, drugs, sugars, sugar alcohols, surfactants, organic acids and bases, inorganic molecules, co-solvents, co-excipients, plasticizers, and combinations thereof.

The polymer used in the amorphous solid dispersion is selected from the group comprising, but not limited to synthetic polymers, natural polymers, nature derived polymers, semi-synthetic polymers, or combinations thereof.

Non-limiting examples of synthetic polymer include polyvinylpyrrolidone homopolymer, poly(vinylpyrrolidone-co-vinyl acetate), crosslinked polyvinylpyrrolidone. polyvinyl caprolactam homopolymer, polyvinyl caprolactam-polyvinyl acetate-polyethylene glycol co-polymers, polyethylene glycol homopolymer, polyvinyl alcohol-polyethylene glycol co-polymers, ethylene oxide-propylene oxide co-polymers, ammonio methacrylate co-polymers, polyacrylic acid, polyacrylic acid co-polymers, polymethacrylic acid homopolymer, polymethacrylic acid co-polymers, polyvinylalcohol homopolymer, polyvinylalcohol co-polymers, polyvinyl acetate phthalate, n-methyl-2-pyrrolidone, bis-vinylcaprolactam, or combinations thereof.

Non-limiting examples of natural polymer and nature-derived polymer include cellulose, starch, chitosan, guar, methylcellulose, carboxymethyl cellulose, carboxymethyl cellulose acetate butyrate, ethyl cellulose, hydroxyethyl cellulose, methylhydroxyethylcellulose, hydroxypropyl cellulose, hydroxypropyl methylcellulose, hydroxypropyl methylcellulose acetate succinate, hydroxypropyl methylcellulose phthalate, cellulose acetate adipate, cellulose acetate adipate propionate, cellulose acetate phthalate, cellulose acetate suberate, cellulose acetate sebacate, 5-carboxypentyl hydroxypropyl cellulose, chitosan hydrochloride, hydroxypropyl-β-cyclodextrins, hydroxypropyl-γ-cyclodextrins, or combinations thereof.

As used herein, the drug used in the amorphous solid dispersion is selected from the group comprising, but not limited to analgesic drugs, anti-inflammatory drugs, antiparasitic drugs, anti-arrhythmic drugs, anti-bacterial drugs, anti-viral drugs, anti-coagulant drugs, anti-cancer drugs, anti-depressant drugs, anti-diabetic drugs, anti-epileptic drugs, anti-fungal drugs, anti-gout drugs, anti-hypertensive drugs, antimalarial drugs, anti-migraine drugs, anti-muscarinic drugs, erectile dysfunction improvement drugs, immunosuppressant drugs, anti protozoal drugs, anti-thyroid drugs, anxiolytic drugs, sedative drugs, hypnotic drugs, neuroleptic drugs, β-blocker drugs, cardiac inotropic drugs, antidiuretic drugs, anti-parkinson drugs, gastro-intestinal drugs, histamine receptor antagonists, lipid regulating drugs, anti-anginal drugs, Cox-2 inhibiting drugs, leukotriene inhibiting drugs, protease inhibitors, muscle relaxants, anti-osteoporosis drugs, anti-obesity drugs, cognition enhancing drugs, anti-urinary incontinence drugs, anti-benign prostate hypertrophy drugs, and combinations thereof.

As used herein, the sugar used in the amorphous solid dispersion is selected from the group comprising, but not limited to mannitol, sorbitol, sucrose, maltose, soluble starches, α-cyclodextrin, β-cyclodextrin, γ-cyclodextrin and combinations thereof.

The suitable surfactant for the use in amorphous solid dispersion of the present invention is selected from the group comprising, but not limited to anionic surfactants, zwitterionic surfactants, amphoteric surfactants, nonionic surfactants, cationic surfactant, and combinations thereof.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are non-limiting examples of anionic surfactants of this type.

Non-limiting examples of suitable anionic surfactants include: sarcosinates, taurates, isethionates, sodium lauryl sulfoacetate, sodium laureth carboxylate, and sodium dodecyl benzenesulfonate. Also suitable are alkali metal or ammonium salts of surfactants such as the sodium and potassium salts of the following: lauroyl sarcosinate, myristoyl sarcosinate, palmitoyl sarcosinate, stearoyl sarcosinate, and oleoyl sarcosinate.

Non-limiting examples of suitable cationic surfactants include derivatives of aliphatic quaternary ammonium compounds having at least one long alkyl chain containing from about 8 to about 18 carbon atoms such as lauryl trimethylammonium chloride, cetyl pyridinium chloride, cetyl trimethylammonium bromide, di-isobutylpherroxyethyl-dimethylbenzylammonium chloride, coconut alkyltrimethylammonium nitrite, cetyl pyridinium fluoride, and blends thereof.

Nonionic surfactants that may be used in the practice of the invention include compounds produced by the condensation of alkylene oxide groups (hydrophilic in nature) with an organic hydrophobic compound which may be aliphatic or alkylaromatic in nature.

Non-limiting examples of suitable zwitterionic surfactants include betaines and derivatives of aliphatic quaternary ammonium compounds in which the aliphatic radicals can be straight chain or branched, and which contain an anionic water-solubilizing group, e.g., carboxy, sulfonate, sulfate, phosphate, or phosphonate.

Non-limiting examples of suitable betaines include: decyl betaine or 2-(N-decyl-N,N-dimethylammonio)acetate, coco betaine or 2-(N-coc-N,N-dimethyl ammonio)acetate, myristyl betaine, palmityl betaine, lauryl betaine, cetyl betaine, stearyl betaine, and blends thereof. The amidobetaines are exemplified by cocoamidoethyl betaine, cocoamidopropyl betaine, lauramidopropyl betaine, and the like.

According to another preferred embodiment of the present application, non-limiting examples of surfactants used in amorphous solid dispersions include benzalkonium chloride (HYAMINE® 1622); Dioctyl sodium sulfosuccinate (DOCUSATE SODIUM), sodium lauryl sulfate (SLS), Polyoxyethylene sorbitan fatty acid ester (Polysorbates, TWEEN & SPAN), polyoxyethylene-polyoxypropylene block copolymers (Poloxamer, PLURONICs, or LUTROLs), polyoxyethylene alkyl ethers (CREMOPHOR A, BRIJ), short-chain glyceryl mono-alkylates or polyoxyethylene fatty acid esters (HODAG, IMWITTOR, MYRJ), d-alpha-tocopheryl polyethylene glycol 1000 succinate (Vitamin E-TPGS™), LIPOSORB® O-20, CAPMUL®, POE-0, polyglycolized glycerides (GELUCIREs); glyceryl PEG 8 caprylate/caprate (LABRASOL), mono- and di-alkylate esters of polyols, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and blends thereof. natural surfactants such as sodium taurocholic acid, 1-palmitoyl-2-oleyl-sn-glycero-3-phosphocholine, lecithin, and other phospholipids and mono- and diglycerides, sucrose fatty acid esters, such as sucrose stearate, sucrose oleate, sucrose palmitate, sucrose laurate, and sucrose acetate butyrate, and the like.

Organic acids useful herein are preferably selected from the group comprising tartaric acid, fumaric acid, succinic acid, citric acid, lactic acid, malic acid, aliphatic sulfonic acids, benzoic acid, ascorbic acid, succinic acid, acetic acid, formic acid, oxalic acid, propionic acid, salicylic acid, gluconic acid, mandelic acid, cinnamic acid, oleic acid, tannic acid, aspartic acid, stearic acid, palmitic acid, glycolic acid, glutamic acid, gluconic acid, glucaronic acid, saccharic acid, isonicotinic acid, methanesulfonic acid, ethanesulfonic acid, p-toluenesulfonic acid, benzenesulfonic acids, or pamoic acid (i.e., 1,1′-methylene-bis-(2-hydroxy-3-naphthoate), or combinations thereof. Non-limiting examples of aliphatic sulfonic acids include methanesulfonic acid, ethanesulfonic acid, isethionic acid or combinations thereof. Non-limiting examples of aromatic sulfonic acids include benzenesulfonic acid, p-toluenesulfonic acid, or combinations thereof.

Organic bases useful herein can be selected from, but not limited to, the group of alkali metal alkoxides, triethylamine, diisopropylamine, diisopropylethylamine (DIPEA), pyridine, 1,8-diazabicyclo[5.4.0]undec-7-ene (DBU), 1,4-diazabicyclo[2.2.2]octane (DABCO) or combinations thereof. Non-limiting examples of alkali metal alkoxides include sodium methoxide, potassium methoxide, potassium tert-butoxide, or combinations thereof.

Non-limiting examples of inorganic molecules used herein include various silica compounds selected from mesoporous silica, silicon dioxide, syloid® 244FP, aerosis® 200 aerosil® R-972, silica gel, or combinations thereof.

According to another preferred embodiment of the present application, the selected ingredients of the amorphous solid dispersion comprise at least one drug and at least one polymer.

As used herein, the experimental results data can include, but is not limited to chemical structure, melting temperature, glass transition temperature of drug, dose, solubility, pKa, and octanol-water partition coefficient (logP).

As used herein, the simulated properties of ingredients of the amorphous solid dispersion, generated using molecular simulation can include, but are not limited to density, free energy, enthalpy of mixing, and solubility parameters.

As used herein, the predicted properties of the amorphous solid dispersions can include, but are not limited to glass transition temperature, physical stability, maximum drug concentration during dissolution in Fasted State Simulating Intestinal Fluid [FaSSIF (Cₘₐₓ)], and drug concentration at 120 min during dissolution in Fasted State Simulating Intestinal Fluid [FaSSIF (C₁₂₀)].

Physical stability of solid dispersions can be predicted employing at least two different temperatures and at least two relative humidity conditions comprising, for example, but not limited to 25° C./60% relative humidity or 40° C./75% relative humidity.

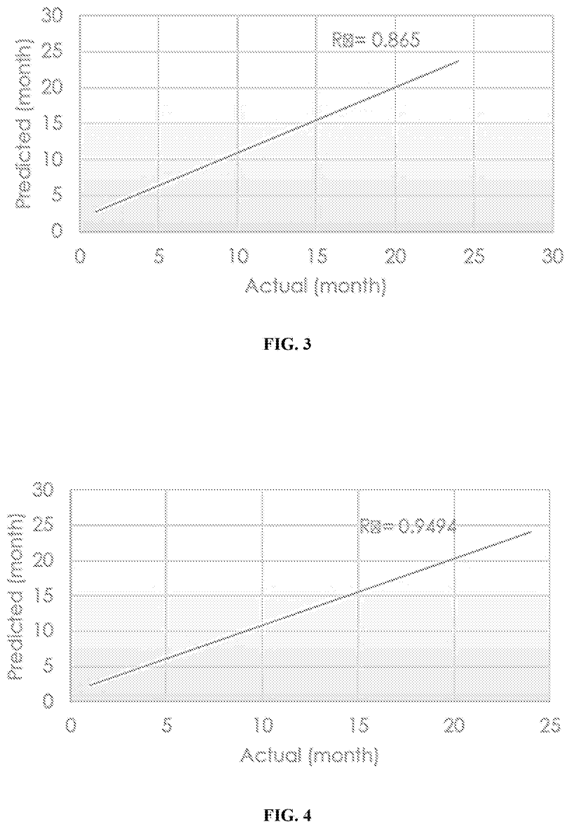

FIG. 3 illustrates a graph representing actual versus predicted values of physical stability at 25° C./60% relative humidity.

FIG. 4 illustrates a graph representing actual versus predicted values of physical stability at 40° C./75% relative humidity.

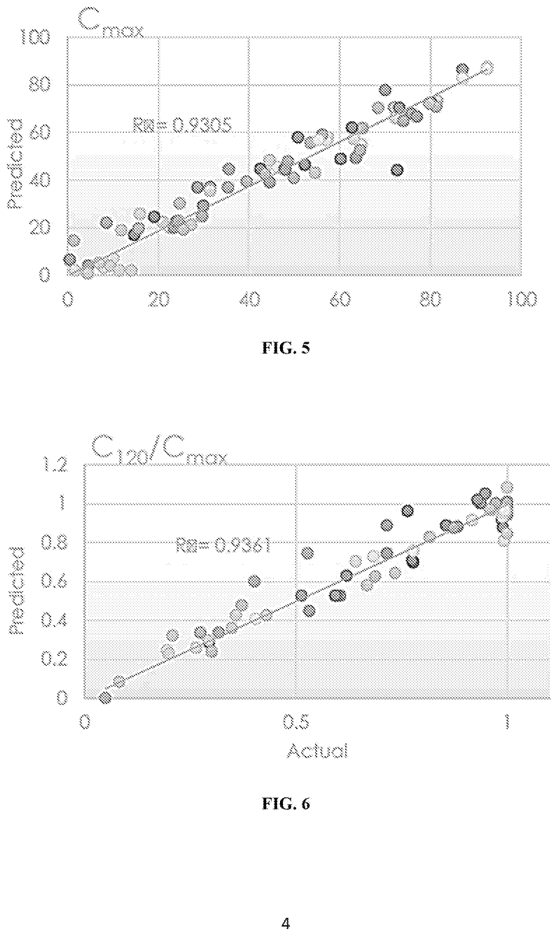

FIG. 5 illustrates a graph representing actual versus predicted values of maximum drug concentration during dissolution in Fasted State Simulating Intestinal Fluid [FaSSIF (Cₘₐₓ)].

FIG. 6 illustrates a graph representing actual versus predicted values of drug concentration at 120 min during dissolution in FaSSIF/Maximum drug concentration during dissolution in FaSSIF (C₁₂₀/Cₘₐₓ).

Another embodiment of the present application relates to a system for predicting properties of amorphous solid dispersions comprising at least one computer system capable of executing the steps of: (i) receiving a plurality of experimental results data of at least one first ingredient of an amorphous solid dispersion; (ii) generating a plurality of two-dimensional or three-dimensional structures of at least one first ingredient of the amorphous solid dispersion of step (i); (iii) performing molecular simulation to generate molecular simulation properties of at least one first ingredient of the amorphous solid dispersion of step (ii); (iv) implementing an artificial neural network using experimental results data of step (i) and molecular simulation properties of step (iii); and (v) predicting the properties of amorphous solid dispersions comprising at least one second ingredient, using the artificial neural network of step (iv).

As used herein, the computer system for predicting properties of amorphous solid dispersions comprises (i) a memory configured to store at least one program. (ii) a processor (iii) a visualization interface, or combinations thereof.

Further, certain aspects of the present application are illustrated in detail by way of the following examples. The examples are given herein for illustration of the application and are not intended to be limiting thereof.

FIG. 7 is a block diagram of an example system architecture of an exemplary device configured to train, store, and/or use a neural network in accordance with one or more implementations. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 700 includes one or more processing units 702 (e.g., microprocessors, ASICs, FPGAs, GPUs, CPUs, processing cores, or the like), one or more input/output (I/O) devices 706, one or more communication interfaces 708 (e.g., USB, IEEE 802.3x, IEEE 802.11x, IEEE 802.16x, GSM, CDMA, TDMA, GPS, IR, BLUETOOTH, ZIGBEE, SPI, I2C, or the like type interface), one or more programming (e.g., I/O) interfaces 710, a memory 720, and one or more communication buses 704 for interconnecting these and various other components. In some implementations, the one or more communication buses 704 include circuitry that interconnects and controls communications between system components.

The memory 720 includes high-speed random-access memory, such as DRAM, SRAM, DDR RAM, or other random-access solid-state memory devices. In some implementations, the memory 720 includes non-volatile memory, such as one or more magnetic disk storage devices, optical disk storage devices, flash memory devices, or other non-volatile solid-state storage devices. The memory 720 optionally includes one or more storage devices remotely located from the one or more processing units 702, The memory 720 comprises a non-transitory computer readable storage medium. In some implementations, the memory 720 or the non-transitory computer readable storage medium of the memory 720 stores the following programs, modules and data structures, or a subset thereof including an optional operating system 730 and one or more modules 740. The operating system 730 includes procedures for handling various basic system services and for performing hardware dependent tasks. The neural network trainer 742 is an example of a module that can be configured to train a neural network according to the techniques disclosed herein. The neural network 744 represents a neural network that has been integrated into an application or otherwise trained and then stored in the memory 720. The simulation engine 746 is an example of a module that can be configured to simulate properties of ingredients as described herein.

FIG. 7 is intended more as a functional description of the various features which are present in a particular implementation as opposed to a structural schematic of the implementations described herein. As recognized by those of ordinary skill in the art, items shown separately could be combined and some items could be separated. The actual number of units and the division of particular functions and how features are allocated among them will vary from one implementation to another and, in some implementations, depends in part on the particular combination of hardware, software, or firmware chosen for a particular implementation.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, resisters, or other information storage devices, transmission devices, or display devices of the computing platform.

Implementations of the methods disclosed herein may be performed in the operation of such computing devices. The order of the blocks presented in the examples above can be varied for example, blocks can be re-ordered, combined, and/or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modifications may be implemented by those skilled in the art without departing from the scope and spirit of the invention.

What is claimed is:

1. A method of predicting properties of amorphous solid dispersions, the method comprising, at an electronic device having a processor:
obtaining a machine learning model trained to predict a dissolution, thermophysical, or stability property of an amorphous solid dispersion based on at least one parameter of at least one first ingredient, wherein the machine learning model is trained based on comparing (a) a predicted dissolution, thermophysical, or stability property predicted based on the at least one first ingredient with (b) an experimentally-determined dissolution, thermophysical, or stability property that is experimentally determined using the at least one first ingredient;
determining at least one parameter of at least one second ingredient of a second amorphous solid dispersion; and
predicting at least one dissolution, thermophysical, or stability property of the second amorphous solid dispersion by inputting the at least one parameter of the at least one second ingredient to the machine learning model.

2. The method according to claim 1, wherein the at least one parameter of the at least one first ingredient comprises at least one molecular simulation property.

3. The method according to claim 1, wherein said at least one parameter of the at least one second ingredient is an experimental active pharmaceutical ingredient (API) parameter, the experimental API parameter comprising a molecular weight, melting point, water solubility, or value associated with an experimental octanol water partition coefficient.

4. The method according to claim 1, wherein said at least one parameter of the at least one second ingredient is a computed active pharmaceutical ingredient (API) parameter, the computed API parameter comprising a number of hydrogen bond acceptors and donors, a solubility value, or a molecular volume or said at least one parameter of the at least one second ingredient is a computed polymer parameter comprising a thermophysical property, a mechanical property, or a geometrical property.

5. (canceled)

6. The method according to claim 1, wherein the machine learning model is trained by:
(i) creating a plurality of experimental results data of the at least one first ingredient of at least one amorphous solid dispersions;
(ii) generating molecular simulation properties of the at least one first ingredient of the amorphous solid dispersion of step (i); and
(iii) training the machine learning model using experimental results data of step (i) and molecular simulation properties of step (ii) wherein said simulated properties comprise density, solvation free energy, enthalpy of mixing, and solubility parameters.

7. (canceled)

8. The method according to claim 1, wherein said at least one first ingredient is selected from the group consisting of polymers, drugs, sugars, sugar alcohols, surfactants, organic acids and bases, inorganic molecules, co-solvents, co-excipients, plasticizers, and combinations thereof.

9. The method according to claim 8, wherein said polymer is selected from the group consisting of homo polymers, co-polymers, oligomers, ampholytic polymers, water soluble polymers, water insoluble polymers, ionizable polymers, non-ionizable polymers and combination thereof or from the group consisting of synthetic polymers, natural polymers, nature derived polymers, semi-synthetic polymers, and combinations thereof.

10. (canceled)

11. The method according to claim 9, wherein said synthetic polymer is selected from the group consisting of polyvinylpyrrolidone homopolymer, poly(vinylpyrrolidone-co-vinyl acetate), crosslinked polyvinylpyrrolidone, polyvinyl caprolactam homopolymer, polyvinyl caprolactam-polyvinyl acetate-polyethylene glycol co-polymers, polyethylene glycol homopolymer, polyvinyl alcohol-polyethylene glycol co-polymers, ethylene oxide-propylene oxide co-polymers, ammonio methacrylate co-polymers, polyacrylic acid, polyacrylic acid co-polymers, polymethacrylic acid homopolymer, polymethacrylic acid co-polymers, polyvinylalcohol homopolymer, polyvinylalcohol co-polymers, polyvinyl acetate phthalate, n-methyl-2-pyrrolidone, bis-vinylcaprolactam, and combinations thereof.

12. The method according to claim 9, wherein said natural polymer and nature-derived polymer are selected from the group consisting of cellulose, starch, chitosan, guar, methylcellulose, carboxymethyl cellulose, carboxymethyl cellulose acetate butyrate, ethyl cellulose, hydroxyethyl cellulose, methylhydroxyethylcellulose, hydroxypropyl cellulose, hydroxypropyl methylcellulose, hydroxypropyl methylcellulose acetate succinate, hydroxypropyl methylcellulose phthalate, cellulose acetate adipate, cellulose acetate adipate propionate, cellulose acetate phthalate, cellulose acetate suberate, cellulose acetate sebacate, 5-carboxypentyl hydroxypropyl cellulose, chitosan hydrochloride, hydroxypropyl-β-cyclodextrins, hydroxypropyl-γ-cyclodextrins, and combinations thereof.

13. The method according to claim 8, wherein said drug is selected from the group consisting of analgesic drugs, anti-inflammatory drugs, antiparasitic drugs, anti-arrhythmic drugs, anti-bacterial drugs, anti-viral drugs, anti-coagulant drugs, anti-cancer drugs, anti-depressant drugs, anti-diabetic drugs, anti-epileptic drugs, anti-fungal drugs, anti-gout drugs, anti-hypertensive drugs, antimalarial drugs, anti-migraine drugs, anti-muscarinic drugs, erectile dysfunction improvement drugs, immunosuppressant drugs, anti-protozoal drugs, anti-thyroid drugs, anxiolytic drugs, sedative drugs, hypnotic drugs, neuroleptic drugs, β-blocker drugs, cardiac inotropic drugs, antidiuretic drugs, anti-parkinson drugs, gastro-intestinal drugs, histamine receptor antagonists, lipid regulating drugs, anti-anginal drugs, Cox-2 inhibiting drugs, leukotriene inhibiting drugs, protease inhibitors, muscle relaxants, anti-osteoporosis drugs, anti-obesity drugs, cognition enhancing drugs, anti-urinary incontinence drugs, anti-benign prostate hypertrophy drugs, and combinations thereof said sugar is selected from the group consisting of mannitol, sorbitol, sucrose, maltose, soluble starches, α-cyclodextrin, β-cyclodextrin, γ-cyclodextrin, and combinations thereof said surfactant is selected from the group consisting of anionic surfactants, cationic surfactants, nonionic surfactants, and combinations thereof.

14. (canceled)

15. (canceled)

16. The method according to claim 1, wherein said at least one first ingredient comprises at least one drug and at least one polymer.

17. The method according to claim 1, wherein said at least one dissolution, thermophysical, or stability property comprises glass transition temperature, physical stability of amorphous solid dispersions, maximum drug concentration during dissolution in Fasted State Simulating Intestinal Fluid [FaSSIF (Cₘₐₓ)], or drug concentration at 120 min during dissolution in Fasted State Simulating Intestinal Fluid [FaSSIF (C₁₂₀)] and preferably, said physical stability of amorphous solid dispersions is predicted employing at least two different temperatures and at least two relative humidity conditions.

18. (canceled)

19. A system for predicting properties of amorphous solid dispersions comprising at least one computer system capable of executing the steps of:
(i) receiving a plurality of experimental results data of at least one first ingredient of an amorphous solid dispersion;
(ii) generating a plurality of two-dimensional or three-dimensional structures of at least one first ingredient of the amorphous solid dispersion of step (i);
(iii) performing molecular simulation to generate molecular simulation properties of at least one first ingredient of the amorphous solid dispersion of step (ii);
(iv) implementing an artificial neural network using experimental results data of step (i) and molecular simulation properties of step (iii); and
(v) predicting the properties of amorphous solid dispersions comprising at least one second ingredient, using said artificial neural network of step (iv).

20. The system according to claim 19, wherein said computer system comprises (i) a memory configured to store at least one program, (ii) a processor, and (iii) a visualization interface, or combinations thereof.

21. The system according to claim 19, wherein said second ingredient is different from said first ingredient of the amorphous solid dispersion, used for creating said experimental results data.

22. The system according to claim 19, wherein said ingredient of the amorphous solid dispersion is selected from the group consisting of polymers, drugs, sugars, sugar alcohols, surfactants, organic acids and bases, inorganic molecules, co-solvents, co-excipients, plasticizers, and combinations thereof.

23. The system according to claim 22, wherein said polymer is selected from the group consisting of homo polymers, co-polymers, oligomers, ampholytic polymers, water soluble polymers, water in-soluble polymers, ionizable polymers, non-ionizable polymers and combination thereof or said polymer is selected from the group consisting of synthetic polymers, natural polymers, nature derived polymers, semi-synthetic polymers, and combinations thereof.

24. (canceled)

25. The system according to claim 23, wherein said synthetic polymer is selected from the group consisting of polyvinylpyrrolidone homopolymer, poly(vinylpyrrolidone-co-vinyl acetate), crosslinked polyvinylpyrrolidone, polyvinyl caprolactam homopolymer, polyvinyl caprolactam-polyvinyl acetate-polyethylene glycol co-polymers, polyethylene glycol homopolymer, polyvinyl alcohol-polyethylene glycol co-polymers, ethylene oxide-propylene oxide co-polymers, ammonio methacrylate co-polymers, polyacrylic acid, polyacrylic acid co-polymers, polymethacrylic acid homopolymer, polymethacrylic acid co-polymers, polyvinylalcohol homopolymer, polyvinylalcohol co-polymers, polyvinyl acetate phthalate, n-methyl -2-pyrrolidone, hydroxyethyl pyrrolidone, bis-vinylcaprolactam, and combinations thereof.

26. The system according to claim 23, wherein said natural polymer or nature-derived polymer is selected from the group consisting of cellulose, starch, chitosan, guar, methylcellulose, carboxymethyl cellulose, carboxymethyl cellulose acetate butyrate, ethyl cellulose, hydroxyethyl cellulose, methylhydroxyethylcellulose, hydroxypropyl cellulose, hydroxypropyl methylcellulose, hydroxypropyl methylcellulose acetate succinate, hydroxypropyl methylcellulose phthalate, cellulose acetate adipate, cellulose acetate adipate propionate, cellulose acetate phthalate, cellulose acetate suberate, cellulose acetate sebacate, 5-carboxypentyl hydroxypropyl cellulose, chitosan hydrochloride, hydroxypropyl-β-cyclodextrins, hydroxypropyl-γ-cyclodextrins, and combinations thereof.

27. The system according to claim 22, wherein said drug is selected from the group consisting of analgesic drugs, anti-inflammatory drugs, antiparasitic drugs, anti-arrhythmic drugs, anti-bacterial drugs, anti-viral drugs, anti-coagulant drugs, anti-cancer drugs, anti-depressant drugs, anti-diabetic drugs, anti-epileptic drugs, anti-fungal drugs, anti-gout drugs, anti-hypertensive drugs, antimalarial drugs, anti-migraine drugs, anti-muscarinic drugs, erectile dysfunction improvement drugs, immunosuppressant drugs, anti-protozoal drugs, anti-thyroid drugs, anxiolytic drugs, sedative drugs, hypnotic drugs, neuroleptic drugs, β-blocker drugs, cardiac inotropic drugs, antidiuretic drugs, anti-parkinson drugs, gastro-intestinal drugs, histamine receptor antagonists, lipid regulating drugs, anti-anginal drugs, Cox-2 inhibiting drugs, leukotriene inhibiting drugs, protease inhibitors, muscle relaxants, anti-osteoporosis drugs, anti-obesity drugs, cognition enhancing drugs, anti-urinary incontinence drugs, anti-benign prostate hypertrophy drugs, and combinations thereof

28. The system according to claim 22, wherein said sugar is selected from the group consisting of mannitol, sorbitol, sucrose, maltose, soluble starches, α-cyclodextrin, β-cyclodextrin, γ-cyclodextrin and combinations thereof.

29. The system according to claim 22, wherein said surfactant is selected from the group consisting of anionic surfactants, cationic surfactants, nonionic surfactants, and combinations thereof.

30. The system according to claim 19, wherein said ingredient of the amorphous solid dispersion comprises at least one drug and at least one polymer.

31. The system according to claim 19, wherein said experimental results data comprise chemical structure, melting temperature, glass transition temperature of drug, dose, solubility, pKa, and octanol-water partition coefficient (logP).

32. The system according to claim 19, wherein said simulated properties comprise density, free energy, enthalpy of mixing, and solubility parameters.

33. The system according to claim 19, wherein said predicted properties comprise glass transition temperature, physical stability, maximum drug concentration during dissolution in Fasted State Simulating Intestinal Fluid [FaSSIF (Cₘₐₓ)], and drug concentration at 120 min during dissolution in Fasted State Simulating Intestinal Fluid [FaSSIF (C₁₂₀)].

34. The system according to claim 19, wherein said physical stability of amorphous solid dispersions is predicted employing at least two different temperatures and at least two relative humidity conditions.
